(19) United States
(12) Patent Application Publication
(10) Pub. No.: US 2004/0076685 A1
(43) Pub. Date: Apr. 22, 2004

(54) Method of preparing porous calcium phosphate morsels and granules via gelatin processing

(75) Inventor: Ahmet Cuneyt Tas, Central, SC (US)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt (DE)

(21) Appl. No.: 10/617,133

(22) Filed: Jul. 11, 2003

(30) Foreign Application Priority Data
Jul. 11, 2002 (EP) 02015436.5

(51) Int. Cl. A61K 33/42; C01B 25/26; C01B 15/16

(52) U.S. Cl. 424/602; 424/603; 423/311

(57) ABSTRACT

The invention describes a method of preparing alpha- or beta-tricalcium phosphate, brushite (CaHPO₄.2H₂O), calcium pyrophosphate (Ca₂P₂O₇) or hydroxyapatite (Ca₅(PO₄)₃(OH)) or mixtures thereof in the form of morsels or granules via gelatin processing. These morsels or granules can be used as bone or tooth fillers or bone substitutes in applications, especially when higher rates of resorption or taking part in the bone remodelling processes of the implanted material are desired.

The invention relates to a method of preparing porous alpha- or beta-tricalcium phosphate (TCP), brushite (CaHPO₄₀.2H₂O), calcium pyrophosphate (Ca₂P₂O₇) or hydroxyapatite (Ca₅(PO₄)₃(OH) or mixtures thereof in the form of morsels (cylinders) or granules via gelatin processing. These morsels or granules can be used as bone or tooth fillers or bone substitutes in applications, especially when higher rates of resorption or taking part in the bone remodeling processes of the implanted material are desired.

As the materials used for artificial bone, artificial tooth and compensation of bones (hereinafter referred to as “bone filler”) in dentistry, cerebral surgery and orthopedic surgery, those materials that are non-toxic, sufficient in mechanical strength, high affinity towards a living body so as to facilitate the direct bonding therewith, and naturally in vivo so as to be naturally replaceable by a newly formed bone are preferred.

As a method for producing such a bone filler with a highly porous structure, it is known to mix a suitable raw material powder with a thermally decomposable material, molding the mixture into a pre-selected form, and performing the removal of the thermally decomposable material and sintering of the raw material powder by consecutive heating (see a.) A. Slosarczyk, “Highly Porous Hydroxyapatite Material,” Powd. Metal. Int., 21, 24-25 (1989), b.) L. Menabue, L. Forti, G. Pellacani, “A Study of Materials Suitable to Produce Bioceramics with Controlled Porosity for Prosthetic Implants Stabilized by Bone Tissue Ingrowth,” Biomaterials, 6, 3-4 (1992), c.) Dean-Mo Liu, “Fabrication and Characterization of Porous Hydroxyapatite Granules,” Biomaterials, 17, 1955-1957 (1996), d.) M. Fabbri, G. C. Celotti, and A. Ravaglioli, “Granulates Based on Calcium Phosphate with Controlled Morphology and Porosity for Medical Applications: Physico-Chemical Parameters and Production Technique,” Biomaterials, 15, 474-477 (1994), e.) E. Ryshkewitch, “Compression Strength of Porous Sintered Alumina and Zirconia” J. Am. Ceram. Soc., 157, 65-68 (1953), f.) N. Passuti, G. Daculsi, J. M. Rogez, S. Martin, and J. V. Bainvel, “Macroporous Calcium Phosphate Ceramic Performance in Human Spine Fusion,” Clin. Orth. Rel. Res., 248, 169-176 (1989), g.) H. S. Byrd, P. C. Hobar, and K. Shewmake, “Augmentation of the Craniofacial Skeleton with Porous HA Granules,” Plast. Reconstr. Surg., 91, 15-26 (1993), h.) J. F. Piecuch, R. G. Topazian, S. Skoly, and S. Wolfe, “Experimental Ridge Augmentation with Porous HA Implants,” J. Dent. Res., 62, 148-154 (1983), i.) Japanese Patent Laid-Open No. 60-21763, j.) Japanese Patent Laid-Open No. 60-16879, and k.) N. O. Engin and A. C. Tas, “Manufacture of Macroporous Calcium Hydroxyapatite Bioceramics,” J. Euro. Ceram. Soc., 19 (13-14), 2569-2572 (1999)).

In these known methods of preparing porous calcium phosphates, however, the contact of the thermally decomposable material added (typically in the form of a solid substance) for formation of pores is not necessarily uniform, and the formed pores are mostly apt to be open cells. Even if the formed adjacent pores are in contact and continued to each other, the sectional area of the communicating part of each pore is minimized. In such a pore structure, it is difficult to make cells necessary for bone formation (osteoblasts and related cells) intrude uniformly into each pore.

Natural bones do basically consist of inorganic calcium phosphate and fibrous organic collagen. Gelatin being the denatured form of collagen, has a significantly high solubility even in water at room temperature. Gelatin, depending on its concentration, may form a viscous, thermo-reversible gel with water, and its use as a biomedical polymer in surgical operations have already been documented (Y. Otani, Y. Tabata, and Y. Ikada, “Adhesion to Soft Tissues by Gelation-Polyanion Hydrogels,” J. Adhesion, 59, 197-205 (1999).

Y. Fujishiro et al., “Preparation and Compressive Strength of alpha-tricalcium phosphate/gelatin gel composite cement,” J. Biomed. Mater. Res., 54, 525-530 (2001), C. H. Yao, C. C. Tsai, Y. S. Chen, C. J. Chang, B. S. Liu, C. C. Lin, and Y. H. Tsuang, “Fabrication and Evaluation of a New Composite Composed of Tricalcium Phosphate, Gelatin and Chi-Li-Saan as a Bone Substitute,” Am. J. Chinese Med., 30, 471-482 (2002), Y. Fujishiro, K. Takahashi, and T. Sato, “Preparation and Compressive Strength of Alpha-Tricalcium Phosphate/Gelatin Gel Composite Cement,” J. Biomed. Mater. Res., 54, 525-530 (2001), M. B. Yaylaoglu, P. Korkusuz, U. Ors, F. Korkusuz, and V. Hasirci, “Development of a Calcium Phosphate-Gelatin Composite as a Bone Substitute and Its Use in Drug Release,” Biomaterials, 20, 711-719 (1999), F. H. Lin, C. H. Yao, J. S. Sun, H. C. Liu, and C. W. Huang, “Biological Effects and Cytotoxicity of the Composite Composed by Tricalcium Phosphate and Glutaraldehyde Cross-linked Gelatin,” Biomaterials, 19, 905-917 (1998), C. H. Yao, J. S. Sun, F. H. Lin, C. J. Liao, and C. W. Huang, “Biological Effects and Cytotoxicity of Tricalcium Phosphate and Formaldehyde Cross-linked Gelatin Composite,” Mater. Chem. Phys., 45, 6-14 (1996), L. Brecevic, V. Hlady, and H. Furedimilhofer, “Influence of Gelatin on the Precipitation of Amorphous Calcium Phosphate,” Colloid Surface, 28, 301-313 (1987), J. M. Rueger, H. R. Siebert, H. Schmidt, and A. Pannike, “Observation on Osteoinduction and Osteostimulation, Released by Bone Gelatin and Its Combination with Beta-Tricalcium Phosphate and Hydroxylapatite Ceramics,” Acta Med. Aust., 13, 27-27 Suppl. 35 (1986), and A. Bigi et al. “Bonelike Apatite Growth on Hydroxyapatite-Gelatin Sponges from Simulated Body Fluid,” J. Biomed. Mater. Res., 59, 709-714 (2002) disclose gelatin as a pore-former in the production of porous calcium phosphate-based biomedical implants.

However, the methods described in these studies implied the use (i.e., implantation) of such calcium phosphate-gelatin composites without a total burnout/removal of the numerous organic amino acids and other substances (which result from the dissolution/decomposition of gelatin in aqueous media in the presence of calcium phosphates).

It must be remembered that gelatin is not such a simple material to start with. Although before its hydrolysis in aqueous media it is a well-defined material, following its dissolution in water (the extent of its dissolution and the exact occurrence of its decomposition products heavily depending on the temperature of solution and its concentration), it turns into a quite complex mixture of organic acids.

The following table (from J. A. Arnesen, et al., Bioresource Technology, 82, 191-194 (2002)) compares the amino-acid compositions of different mammalian gelatins in hydrolyzed gelatin samples, whereas the numbers denote “moles per 100 mole of amino acids.” Therefore, utmost caution must be exercised while the use of gelatin sponges mixed with calcium phosphates as a direct implantation material (without a thermal decomposition/burn-out step) is considered.

Amino

| Amino acid | Porcine | Bovine | Whale |
|---|---|---|---|
| Glycine | 30.8 | 33.3 | 30.2 |
| Proline | 12.7 | 12.4 | 10.8 |
| Alanine | 11.1 | 11.5 | 10.4 |
| Hydroxyproline | 10.9 | 9.6 | 8.5 |
| Glutamic acid | 7.8 | 7.4 | 8.0 |
| Arginine | 5.1 | 4.6 | 5.3 |
| Aspartic acid | 4.4 | 4.3 | 4.8 |
| Serine | 3.3 | 3.2 | 4.0 |
| Lysine | 2.7 | 2.6 | 3.0 |
| Leucine | 2.6 | 2.4 | 2.8 |
| Valine | 2.3 | 2.0 | 2.2 |
| Threonine | 1.8 | 1.7 | 2.9 |
| Phenyalanine | 1.3 | 1.3 | 1.5 |
| Isoleucine | 1.1 | 1.2 | 1.2 |
| Hydroxlysine | 0.7 | 0.7 | 0.9 |
| Methionine | 0.5 | 0.5 | 0.6 |
| Histidine | 0.4 | 0.5 | 0.6 |
| Ornithine | 0.2 | 0.6 | 0.0 |
| Tyrosine | 0.2 | 0.1 | 0.5 |

Many of these amino-acids, at the levels indicated in the above table, when incorporated in an implant material must be thoroughly tested for the presence of any adverse or side effects on the implant site. In other words, the presence of such organic acids may readily alter the expected bone-healing behavior of the calcium phosphates used together with them.

A feature of the present invention is to provide a method for preparing porous alpha- or beta-TCP, brushite (CaHPO4(2H2O), calcium pyrophosphate (Ca2P2O7) or hydroxyapatite (Ca5(PO4)3(OH)) or mixtures thereof in the form of morsels or granules via gelatin processing, which avoids the above-mentioned disadvantages from the prior art.

Upon further study of the specification and appended claims, further features and advantages of this invention will become apparent to those skilled in the art.

These features can be achieved by a method of preparing porous alpha- or beta-TCP, brushite (CaHPO4(2H2O), calcium pyrophosphate (Ca2P2O7) or hydroxyapatite (Ca5(PO4)3(OH)) or mixtures thereof in the form of morsels or granules via gelatin processing characterized in that the method comprising the steps of:

a) mixing a calcium phosphate self-setting cement powder and gelatin powder in a weight ratio of e.g., 3:0.25 to 1;

b) adding Na2HPO4 solution followed by mixing paste formed;

c) shaping the paste, preferably immediately, in, e.g., a device for drawing in and ejecting the paste, such as an injection molding apparatus, an extruder or a syringe;

d) placing the shaped paste in a solvent, e.g. in distilled water at e.g., 37° C. for, e.g. a few days, to dissolve away gelatin and to form interconnected pores;

e) thermally treating to burnout all organic or volatile material followed by successive cooling to room temperature; and

f) optionally crushing the calcined, sintered calcium phosphate material and then sieving to obtain porous granules.

According to the invention the following starting materials (calcium phosphates) are preferred:

a) Chemically synthesized α-TCP powder (where TCP is Ca₃(PO₄)₂ with a Ca/P molar ratio of 1.50),

b) Chemically synthesized β-TCP powder,

c) Chemically synthesized “bi-phasic” mixtures of HA and α-TCP powder (where HA is Ca₅(PO₄)₃(OH) with a Ca/P molar ratio in the range of 1.51 to 1.65,

d) Chemically synthesized DCPD powder (dicalcium phosphate dihydrate CaHPO₄.2H₂O with Ca/P=1.00),

e) DCPA powder (dicalcium phosphate anhydrous, CaHPO₄ with Ca/P=1.00 obtained by heating DCPD powders (of item d above) at 120° C.), or

f) Chemically synthesized ACP powder (amorphous calcium phosphate, with Ca/P being variable over the range of 0.8 to 1.60).

The above-mentioned calcium phosphate powders when mixed with one another in proper ratios will result in self-setting calcium phosphate cements.

Some exemplary calcium phosphate cements are disclosed in:

1) K. Kurashina, H. Kurita, M. Hirano, A. Kotani, C. P. A. T. Klein, and K. de Groot, “In Vivo Study of Calcium Phosphate Cements: Implantation of an α-Tricalcium Phosphate/Dicalcium Phosphate Dibasic/Tetracalcium Phosphate Monoxide Cement Paste,” Biomaterials, 18, 539-543 (1997).

2) B. R. Constantz, B. M. Barr, I. C. Ison, M. T. Fulmer, D. C. Delaney, J. Ross, and R. D. Poser, “Histological, Chemical and Crystallographic Analysis of Four Calcium Phosphate Cements in Different Rabbit Osseous Sites,” J. Biomed. Mater. Res (Appl. Biomater.), 43, 451-461 (1998).

3) D. Knaack, M. E. P. Goad, M. Aiolova, C. Rey, A. Tofighi, P. Chakravarthy, and D. D. Lee, “Resorbable Calcium Phosphate Bone Substitute,” J. Biomed. Mater. Res. (Appl. Biomater.),43, 399-409 (1998).

4) F. C. M. Driessens, M. G. Boltong, E. A. P. de Maeyer, R. M. H. Verbeeck, and R. Wenz, “Effect of Temperature and Immersion on the Setting of Some Calcium Phosphate Cements,” J. Mater. Sci. Mater. Med., 11, 453-457 (2000).

5) E. M. Ooms, J. G. C. Wolke, J. P. C. M. van der Waerden, and J. A. Jansen, “Trabecular Bone Response to Injectable Calcium Phosphate (Ca—P) Cement,” J. Biomed. Mater. Res., 61, 9-18 (2002).

6) O. Gauthier, I. Khairoun, J. Bosco, L. Obadia, X. Bourges, C. Rau, D. Magne, J. M. Bouler, E. Aguado, G. Daculsi, and P. Weiss, “Noninvasive Bone Replacement with a New Injectable Calcium Phosphate Biomaterial,” J. Biomed. Mater. Res., 66A, 47-54 (2003).

Mixing ratios of the powders listed above will give the user a freedom in adjusting the Ca/P molar ratio of the final powder body. Some of those powders are not self-setting cements by themselves, but upon mixing with one another they become one. The use of self-setting calcium phosphate cement formulations, as a starting material, provides the unique ability to easily impart any desired shape to the final product, which shall not just be restricted to the shape of small cylinders/morsels.

Following table, for example, will explain the mixing order and percentages of the individual powders to yield self-setting cements:

| | | | Ca/P |
|---|---|---|---|
| | | | ratio range |
| | | Mixing ratio | to be |
| Powder | | (weight fraction) | attained |
| 1 | A | 1 | 1.50 |
| 2 | A + B | ¼B + ¾A | 1.50 |
| 3 | C | 0.05 HA + 0.95 α-TCP | 1.54-1.55 |
| 4 | C + D | ¾C + ¼D | 1.39 |
| 5 | A + C + E + CaCO₃ | 0.63 + 0.02 + 0.25 + 0.10 | 1.51-1.52 |
| 6 | F + D | 0.60 to 0.80 F + 0.40 to 0.20 D | 1.00 to 1.50 |

Each one of these 6 powders given in the above table can be taken as the starting self-setting calcium phosphate cement, and then these powders are to be mixed with proper amounts, 3:0.25-3:1, preferably 3:0.7-3:1, by weight of cement powder to gelatin, of gelatin (Merck KGaA, gelatin powder food grade, Cat. Nr. 104078) by ball milling or by hand mixing in an agate mortar with an agate pestle. Generally, preferred gelatins are those that provide good results in term of porosity, pore sizes and pore morphology. Maintaining the same bloom number will provide more or less the same gelling properties even when using different gelatins. Exemplary gelatin sources include bovine, e.g., demineralized bone or cowhide, porcine, e,g, pigbone or pigskin, donkey hide, fish, e.g., tuna skin, and/or whale. Generally, gelatin sources do not differ significantly from one another in solubility, e.g., in lukewarm water, and in burn off behavior, e.g., upon heating to high temperatures.

After adding Na₂HPO₄ and squeezing out the morsels, there are two processing possibilities from this point onwards, namely, step 1.), then 2.) can be conducted, alternatively, only step 2.) can be conducted:

1.) If the formed morsel needs further machining (i.e., cutting, slicing, drilling, etc.), the morsel can be dried, e.g., at room temperature for around 24-48 hours, preferably 36-48 hours and optimally 48 hours. When the morsel reaches a compressive strength of around 20 MPa, i.e. sufficient mechanical strength to withstand machine handling, and it can then be machined.

2.) If the morsel does not require any machining, it can be placed directly in distilled water at e.g., room temperature, or alternatively, generally 20′-50° C., preferably 25°-40° C., and optimally 37° C. for about 24-72 hours, preferably 36-60 hours, and optimally 48 hours, after removing it out of the syringe.

These two processing possibilities are applicable for TCP, brushite, pyrophosphate and hydroxyapatite.

The second route is desired, because the “soaking-in-water” step will dissolve away gelatin and will form interconnected pores. Amino acids formed during the dissolution of gelatin component will also result in the local dissolution (in the micron-levels) of the calcium phosphate matrix, and create a communicating network of micropores around the macropores generated by the leached out gelatin particles. The sizes of the formed macropores essentially depend on the initial particle size of gelatin powder used, which was around 250 to 400 μm.

After the proper choice of one of the above-mentioned possibilities (dictated by the product specifications, i.e., slices, holes to be drilled, oblique angles on one end of the morsel, etc.), both kind of samples will receive the same thermal treatment to burnout all the organic or volatile material.

When heated alone in air, gelatin totally volatilizes at around 750° C. However, when the total burnout of gelatin is achieved at that temperature, the remaining porous skeleton of calcium phosphate does not have the required mechanical stability to be handled, and for that reason the thermal treatment temperature preferably approaches the sintering temperature of the calcium phosphate compound under consideration. For all powders, general effective sintering temperatures are 1200-1300° C., preferably 1200-1225° C. For specific powders, the optional sintering temperature for TCP is 1200° C., while the optional sintering temperature for other powders, e.g., brushite, pyrophosphate or hydroxyapatite, is 1250° C. With respect to all materials, sintering of the morsels permits formation of interconnected micro pores (by raising the temperature slightly higher then the actual organic burn off temperature of the gelatin) and increases the mechanical strength of the morsels.

For example, sintering of morsels dried at 37° C. can be conducted in an electrically-heated chamber furnace on Al₂O₃ flat plates, and then heated from room temperature (RT) to sintering temperature, e.g., 1200° C. for TCP in about 500 minutes, soaked e.g., at 1200° C. for 360 minutes, followed by cooling to RT. If the morsels are quenched, e.g., lowered from 1200° to 1000° C. in 10 minutes, the high-temperature polymorphic form of TCP can be brought down to RT, and the product will consist of single-phase α-TCP. If the morsels are slowly cooled within the furnace (e.g., from 1200° C. to RT in 6 hours), then the samples will be single-phase β-TCP. Intermediate cooling rates (i.e., between those of quenching and slow cooling, e.g., cooling from 1200° to 1000° C. in 1 h) will result in the formation of bi-phasic (i.e., almost equimolar mixtures of α- and β-TCP phases) TCP materials. Since the beta form is more resorbable as compared to the alpha form, the control (in terms of thermal treatment regimes utilized) to be gained over the mixing ratio of these two phases in the final product will provide a quite useful tool in tailoring in the in vivo resorption rates of these morsels.

Also, distinct phases can be created in Powder 6 of Table 1 at page 6. Particularly, slow cooling (as, e.g., using substantially the same cooling procedure hereinafter discussed in Example 1) can create small quantities of a hydroxyapatite phase that would crystallize in situ if the Ca/P molar ratio is 1.

Calcium phosphate cement powder is preferably mixed with gelatin powder in a mixing ratio by weight of 3:0.25-3:1, most preferably 3:0.7-3:1. Alternatively, the weight of calcium phosphate cement powder to gelatin powder is generally 3.0:0.25-3.0:2.0; preferably, 3.0:0.6-3.0:1, and optimally 3.0:0.68-3.0:0.72.

The most preferred range of total porosity to be achieved (without destroying the cylindrical morsel or any other initially intended geometrical shape) is 35 to 50%.

Similarly, the initial particle size distribution possessed by the gelatin powder (e.g., porcine) strongly affect the sizes of the macropores to be attained in the final products. The gelatin powder can have 45% e.g., dry particles in the range of 250 to 700 μm (with the average particle size in this range being observed at around 400 μm), and the rest are generally smaller than 250 μm. Average of the macropores observed in the calcined products are in the range of 300 to 400 μm, whereas the average of micropores are generally observed in the range of 3 to 5 μm. All macropores are connected to one another with the micropores.

A self-setting cement is a special (or appropriate) mixture of more than one component (except of α-TCP which is by itself a low-strength self-setting cement) of calcium phosphate compounds to be selected either from the binary system of CaO—P₂O₅ or from the ternary system of CaO—P₂O₅—H₂O, which starts to set when mixed with a small amount of pure water (and more preferably, when mixed with a small amount of water which contains small amounts (1 to 4 wt %) of a basic phosphate compound, such as Na₂HPO₄).

The morsels are preferably cylinders with diameters variable within the range of 0.5 to 2.5 cm, most preferably of 1 cm diameter, and having a height of 1 to 4 cm.

The granules are preferably irregularly shaped particles with a granule size distribution easily adjustable (achieved by sieving) over the range of 0.5 to 5 mm.

The invention is described in detail below in terms of the following working examples.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### Example 1

Production of Porous Morsels of Ca₃(PO₄)₂: (“Injection molding”)

30 grams of α-TCP powder (powder A) and 10 grams of Gelatin powder are mixed in a plastic bottle in a Turbula mill for 1 hour. Then, a 4.0 grams portion of this mixture is placed into an agate mortar. 2.5 mL of 3 wt % aqueous Na₂HPO₄.2H₂O solution (or 2.75 mL of 2 wt % Na₂HPO₄.2H₂O solution) is added to the mortar with a pipette, followed by mixing the formed paste with an agate pestle for 30 seconds. The formed paste (mainly with the immediate chemical reaction taking place between the basic (pH>9) setting solution and gelatin) is immediately placed into a 5 mL syringe within around 2 minutes after the mixing of the powder and setting solution described above, and the morsel is squeezed out of the syringe after 10 minutes. The procedure described here can be named as the injection molding of a viscous paste of calcium phosphate+gelatin mixtures. The mold can therefore have any geometrical shape as desired.

There are two processing possibilities from this point onwards:

1.) If the formed morsel needs further machining (i.e., cutting, slicing, drilling, etc.), the morsel generally must be kept dry at room temperature for around 2 days. Within 2 days the morsel reaches a compressive strength of around 20 MPa, and it can then be machined.

2.) If the morsel does not require any machining, it can be placed directly in distilled water at 37° C., for 2 days, after removing it out of the syringe.

The second route is the most preferred one.

Then, the thermal treatment to burnout all the organic or volatile compounds follows.

When heated alone in air, gelatin totally volatilizes at about 750° C. The thermal treatment temperature must be pushed upwards until the sintering temperature (i.e. for TCP=1200° C.).

Morsels dried at 37° C. prior to the thermal treatment are placed into an electrically-heated chamber furnace on Al₂O₃ flat plates, and then heated from RT to 1200° C. in 500 minutes, and soaked at 1200° C. for 360 minutes, followed by cooling to RT. At this point arised two more possibilities for the producer, if the morsels are quenched from 1200° to 1000° C. in 10 minutes, the high-temperature polymorphic form of TCP can be brought down to RT, and the product will consist of single-phase α-TCP, and if the morsels are slowly cooled within the furnace (from 1200° C. to RT in 6 hours), then the samples will be single-phase β-TCP. Intermediate cooling rates (i.e., between those of quenching and slow cooling, e.g., cooling from 1200° to 1000° C. in 1 h) will result in the formation of bi-phasic TCP materials.

### EXAMPLE 2

Production of Porous Granules:

Calcined, sintered morsels are crushed, and then sieved with a series of the following sieves of respective opening sizes: 5 mm, 2.8 mm, 2 mm, 1.25 mm and 1 mm. Granules formed in this way possess irregular shapes. However, prolonged sieving has the proven tendency to round off the sharp edges of those.

### EXAMPLE 3

Evaluation of the Porosity and Pore Size Distribution in Morsels or Granules:

Total porosity in the produced morsels or granules are directly determined by the density measurements, based on a gas-absorption technique. The theoretical density of the calcium phosphate compounds are well known and do only slightly vary from one another over the range of 3.1 to 3.2 g/cm³. Experimentally measured densities of each sample are divided by the theoretical density of the specific phase comprising the sample is made out of, and multiplied by 100. The resultant number, when subtracted from 100 gives the total porosity in the samples. Percentage of total porosity is then reported on a statistical average basis for that batch of samples. Pore size measurements are performed by using a scanning electron microscopy (SEM) on Au-Pd coated (20-50 angstrom) samples. Macropore and micropore sizes are then directly measured from the enlarged SEM photomicrographs. Density measurements and SEM analysis are performed both on morsels and granules. Crushing of the morsels to form granules does not change the pore size distribution in the granules (as compared to the mother morsels).

### EXAMPLE 4

Control of Porosity and Pore Size Distribution in Morsels and Granules:

The amount of gelatin powder initially blended with the calcium phosphate powders strongly influence the total porosity in the final products. Gelatin powder is most preferably to be mixed with 3 grams of calcium phosphate powder over the range of 0.25 to 1 g. When the gelatin amount is increased beyond 1 g (up to 2 g), consecutive soaking of the formed morsels in water leads to the losing of the formed shape. The most preferred range of gelatin addition to 3 g of calcium phosphates is 0.7 to 1 g. The most preferred range of total porosity to be achieved by this technique (without destroying the cylindrical morsel or any other initially intended geometrical shape) is 35 to 50%. Interconnecting pores are dynamically formed within the first half hour during the soaking of the forms in water. The forms must not be kept in water for more than 3 hours, in order not to destroy their shapes. The undissolved portion of the gelatin is removed during the calcination/sintering step.

### EXAMPLE 5

Production of Porous Morsels with a Phase Mixture of Ca₂P₂O₇ and Ca₃(PO₄)₂:

22.47 grams of powder C (a bi-phasic (95%-5%) mixture of α-TCP and calcium hydroxyapatite, HA), 7.53 g powder D (CaHPO₄.2H₂O) and 10 grams of Gelatin powder are mixed in a plastic bottle in a Turbula mill for 1 hour. Then, a 4.0 grams portion of this mixture is placed into an agate mortar. 2.5 mL of 3 wt % aqueous Na₂HPO₄.2H₂O solution (or 2.75 mL of 2 wt % Na₂HPO₄.2H₂O solution) is added to the mortar with a pipette, followed by mixing the formed paste with an agate pestle for 30 seconds. The formed paste (mainly with the immediate chemical reaction taking place between the basic (pH>9) setting solution and gelatin) is immediately placed into a 5 mL syringe within around 2 minutes after the mixing of the powder and setting solution described above, and the morsel is squeezed out of the syringe after 10 minutes. The formed morsel is soaked in distilled water at 37° C. for 2 days, followed by drying at 60° C. overnight. The morsels produced in this way are then heated in an air atmosphere to 1250° C. in 500 min, kept at that temperature for 6 hours, and then cooled to RT within the electrically-heated chamber furnace in 6 hours. X-ray diffraction analysis performed on the samples indicated the presence of 30 to 35% Ca₂P₂O₇ and 65 to 70% β-TCP. Owing to the initial Ca/P molar ratio of 1.39 utilized in the starting powder mix, these porous morsels are expected to show better resorption characteristics as compared to those made out of only TCP. The granules of this material are easily prepared by crushing and sieving the above.

The entire disclosure of all applications, patents and publications, cited herein and of corresponding EP Application No. 02015436.5, filed Jul. 11, 2002, is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method of preparing porous alpha- or beta-tricalcium phosphate, brushite (CaHPO₄.2H₂O), calcium pyrophosphate (Ca₂P₂O₇) or hydroxyapatite (Ca₅(PO₄)₃(OH)) or mixtures thereof in the form of morsels or granules comprising:
a) mixing a calcium phosphate self-setting cement powder and gelatin powder in a ratio effective to create subsequently formed pores;
b) adding a Na₂HPO₄ solution followed by mixing the formed paste;
c) placing the formed paste into a device;
d) squeezing out the morsels from the device;
e) placing the morsels, after removing them from the device in distilled water at a time and temperature effective to dissolve away the gelatin and to form interconnected pores;
f) thermally treating to burnout all organic or volatile material followed by successive cooling to room temperature; and
g) optionally crushing calcined, sintered morsels and then sieving to obtain porous granules.

2. A method according to claim 1, further comprising, after squeezing the morsels:
d₁) keeping dry the formed morsels at room temperature for about 2 days for further machining.

3. A method according to claim 1, wherein the mixing ratio is 3:0.7-3:−1.

4. A method according to claim 1, wherein the thermal treatment temperature is up to the sintering temperature of the respective calcium phosphate compound.

5. A method of preparing porous alpha- or beta-tricalcium phosphate, brushite (CaHPO₄.2H₂O), calcium pyrophosphate (Ca₂P₂O₇) or mixtures thereof, comprising:
thermally treating a morsel made by mixing a calcium phosphate cement powder and a gelatin powder to a sintering temperature to burnout all organic or volatile material.

6. A method according to claim 5, wherein thermally treating the morsel is conducted at a temperature effective to burnout the morsel.

7. A method according to claim 6, wherein the effective temperature is at least 1200° C.

8. A method according to claim 1, further comprising after thermally treating, crushing calcined, sintered morsels and then sieving to obtain porous granules.

9. A method according to claim 1, wherein the calcium phosphate comprises α-TCP, β-TCP, Ca₅(PO₄)₃(OH), dicalcium phosphate dihydrate, anhydrous dicalcium phosphate, or amorphous calcium phosphate.

10. A method according to claim 5, further comprising, before thermally treating, placing morsels in distilled water at a temperature and duration effective to form interconnected pores.

11. A method according to claim 10, wherein the temperature is 37° C. and the duration is 2 days.

12. A method according to claim 1, wherein the pores have a size of 250-400 microns.

13. A method according to claim 1, wherein the cooling after thermal treating comprises quenching at a time and temperature effective to obtain a single-phase α-TCP.

14. A method according to claim 1, wherein the cooling after thermal treating comprises cooling at a time and temperature effective to create a single-phase β-TCP.

15. A method according to claim 13, wherein the cooling after the thermal treating comprises quenching from 1200° C. to 1000° C. in about 10 minutes to obtain a single-phase α-TCP.

16. A method according to claim 14, wherein the cooling after thermal treating comprises slowly cooling from 1200° C. to 1000° C. in about 1 hour to obtain a single-phase β-TCP.

17. A method according to claim 1, wherein the ratio of cement powder and gelatin powder is 3:0.25-3:1 and the time and temperature effective to dissolve away the gelatin and to form the interconnected pores are, 37° C. and a few days.

18. A method according to claim 17, wherein the time is at least three days.

19. A method of preparing a porous calcium phosphate material of alpha- or beta-tricalcium phosphate, brushite (CaHPO₄.2H₂O), calcium pyrophosphate (Ca₂P₂O₇) or hydroxyapatite (Ca₅(PO₄)₃(OH)) or mixtures thereof, comprising:
shaping a mixture of a calcium phosphate cement powder and gelatin powder in a ratio of 3:0.25-3:1;
soaking a shaped mixture in a solvent at a time and temperature effective to leach out the gelatin and form interconnected pores; and
thermally treating the shaped mixture at a temperature effective to burnout any organic or volatile material.
